(19) United States
(12) Patent Application Publication
(10) Pub. No.: US 2025/0129424 A1
(43) Pub. Date: Apr. 24, 2025

(54) ISOLATION OF FETAL NEURONAL EXTRACELLULAR VESICLES

(71) Applicant: Research Foundation for Mental Hygience, Inc., Menands, NY (US)

(72) Inventor: Nanbert Zhong, Staten Island, NY (US)

(73) Assignee: Research Foundation for Mental Hygience, Inc., Menands, NY (US)

(21) Appl. No.: 18/912,762

(22) Filed: Oct. 11, 2024

Related U.S. Application Data
(60) Provisional application No. 63/544,810, filed on Oct. 19, 2023.

(51) Int. Cl. C12Q 1/6883; G01N 33/543; G01N 33/68

(52) U.S. Cl. CPC C12Q 1/6883; G01N 33/54313; G01N 33/6896; G01N 2333/471

(57) ABSTRACT

A two-step immunoprecipitation technique for isolating fetal neuronal extracellular vesicles (EVs) including exosomes (EXs). First, an antibody against pregnancy-specific beta-1-glycoprotein 1 (PSG1) is used to isolate fetal-originated EVs-EXs from placental syncytiotrophoblasts to obtain placenta-specific EVs-EXs that are of fetal origin circulating in maternal blood. Next, an antibody of synaptic vesicle glycoprotein 2B (SV2B) is used to purify neuronal EVs-EXs from the placental EVs-EXs. The SV2B-immunoprecipitated EVs-EXs according to the present invention may be used for quantitative measurement to determine synaptic function and synapse development, which are related to brain developmental disabilities.

### CROSS-REFERENCE TO RELATED APPLICATIONS

The present application claims priority to US Provisional Application No. 63/544,810 filed on Oct. 19, 2023.

### BACKGROUND OF THE INVENTION

### 1. Field

The present disclosure relates to extracellular vesicles and, more specifically, to

an approach for isolating fetal neuronal extracellular vesicles for prenatal assessment of intrauterine neuronal development.

### 2. Description of the Related Art

Maternal COVID-19 infection during pregnancy exposes the fetus to SARS-COV-2 and may adversely affect fetal brain development. Extracellular vesicles (EVs), particularly exosomes (EXs), released by various cells, provide a promising avenue for studying fetal brain development. For example, isolated placental EVs-EXs, which contain synaptic vesicle-associated transcripts (SVATs), from maternal blood have been studied to evaluate the pathogenic impact of pregnancies on fetal brain development. Certain gene differential expression profiles for SVATs may be associated with autism, attention deficit hyperactivity disorder, and non-syndromic intellectual disability.

However, comprehending this impact is hindered by the challenge of obtaining fetal brain specimens. During pregnancy, EVs-EXs circulating in maternal blood are primarily secreted by the placenta, with a smaller portion originating from the developing fetus. The isolation directly from maternal blood of fetal EVs-EXs, particularly those derived from fetal neurons holds immense promise for assessing fetal brain development and predicting the likelihood of developmental disabilities (DDs). However, isolation of fetal neuron EVs-EXs is challenging because EVs-EXs are secreted as a mixture of different types of cell-originated with contamination of maternal EVs-EXs and EVs-EXs of non-neuronal cell types, rather than secreted only from fetal cells, particularly fetal neurons. Obtaining biological specimens from the intrauterine fetus during pregnancy thus presents a significant challenge when it comes to assessing fetal development, particularly that of the fetal brain. Accordingly, there is a need in the art for an approach for isolating fetal neuron EVs-EXs from material blood that is non-invasive, more specific, and more sensitive than conventional approaches.

### BRIEF SUMMARY

The present invention provides a technique for non-invasively isolating fetal neuron EVs-EXs from maternal blood that can provide sufficient quantities for the performance of quantitative measurements to determine synaptic function and synapse development. More specifically, the present invention employs a two-step immunoprecipitation technique for isolating fetal neuronal EVs-EXs, thereby enabling a quantification of the molecules encapsulated within those vesicles. The two-step procedure involves first using an antibody against pregnancy-specific beta-1-glycoprotein 1 (PSG1) to isolate fetal-originated EVs-EXs from placental syncytiotrophoblasts to obtain placenta-specific EVs-EXs that are of fetal origin circulating in maternal blood. Next, a second antibody is used to purify neuronal EVs-EXs from the placental EVs-EXs. This antibody is specifically against SV2B, a member of the synaptic vesicle proteins 2 (SV2) family that is localized to synaptic vesicles and may function in the regulation of vesicle trafficking and exocytosis in neuron cells. The antibody specific to synaptic vesicle glycoprotein 2B (SV2B) may comprise rabbit polyclonal IgG anti-human/mouse SV2B antibodies conjugated with biotin. Separating the neuronal extracellular vesicles from the placental extracellular vesicles may include adding a beaded resin that binds any biotinylated proteins. Isolating any fetal-originated extracellular vesicles in the blood sample may include using a cell lysis reagent containing protease and phosphatase inhibitors to provide an amount of purified fetal neural extracellular vesicles.

The present invention also provide for a method of monitoring fetal brain development. First, fetal neural extracellular vesicles are isolated from a sample of maternal blood, such as by treating the sample of material blood with an amount of antibodies specific to pregnancy-specific beta-1-glycoprotein 1 (PSG1) to obtain isolated fetal-originated extracellular vesicles and then separating any neuronal extracellular vesicles from any placental extracellular vesicles in the isolated fetal-originated extracellular vesicles by treating the isolated fetal-originated extracellular vesicles with an amount of antibodies specific to synaptic vesicle glycoprotein 2B (SV2B). Next, a marker of the isolated fetal neural extracellular vesicles may be assessed. Finally, the steps may be repeated to assess any changes in the marker of the fetal neural extracellular vesicles over a predetermined period of time, such as from the first trimester to birth. The marker may be an observable characteristic or an amount of expression of a gene, such as the vesicular inhibitory amino acid transporter (VGAT) gene, the vesicular glutamate transporter 2 (VGLUT2) gene, the L1 cell adhesion molecule (L1CAM) gene, the synaptotagmin 1 (SYT1) gene, the synaptic vesicle glyocprotein 2 (SV2B) gene, or the contactin 2 (CNTN2) gene.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a flowchart of a process for immunoprecipitation of isolating fetal neuronal EVs-EXs according to the present invention.

FIG. 2 is a series of graphs of ASD-associated SVATs quantitatively measured in placental EVs-EXs.

FIG. 3 is a series of images of confocal fluorescent staining of EVs-EXs. Anti-CD63 antibody stained placental total EVs-EXs and stained SV2B-immunoprecipitated fetal neural EVs-EXs.

FIG. 4A through 4E are a series of image of confocal fluorescent double-staining, where FIG. 4A is total placental EVs-EXs contain neuronal EVs-EXs (CD63 overlapped with SV2B), FIG. 4B is total placental EVs-EXs contain neuronal EVs-EXs (CD63 overlapped with L1CAM), FIG. 4C is SV2B Immunoprecipitated EVs-EXs contain neuronal EVs-EXs (SV2B overlapped with L1CAM), FIG. 4D is SV2B Immunoprecipitated EVs-EXs contain neuronal EVs-EXs (SV2B overlapped with CNTN2), and FIG. 4E is SV2B Immunoprecipitated EVs-EXs may be isolated at stage of as early as TUBB3 expressed (SV2B overlapped with TUBB3).

FIG. 5 is a graph of the expression of neuronal marker genes (X axis), which are associated with developmental disabilities, may be quantified with ELISA in normal (blue bar) or COVID-infected (orange bar) pregnancies.

FIG. 6 is a series of graphs of the impact of COVID on neuronal signals of placental EVs-EXs showing vesicular inhibitory amino acid transporter (VGAT) and vesicular glutamate transporter 2 (VGLUT2) that were measured in placental EV-EXs with ELISA from the first to the third trimester between normal and COVID-infected pregnancies.

### DETAILED DESCRIPTION

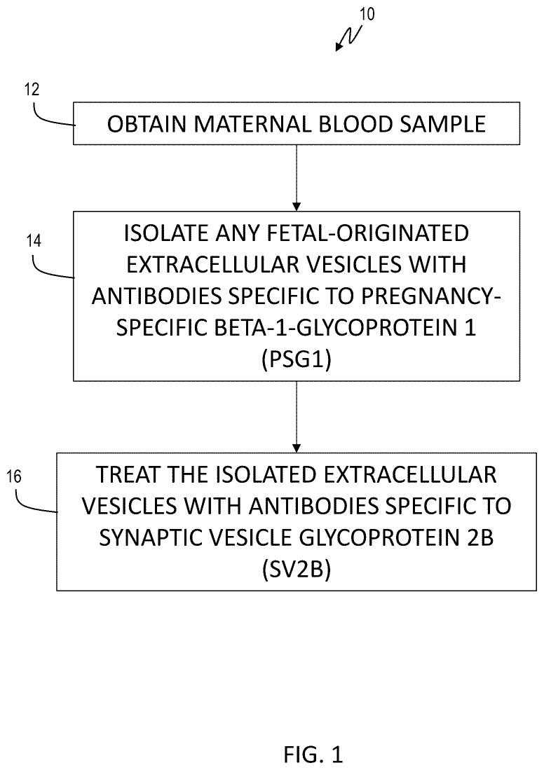

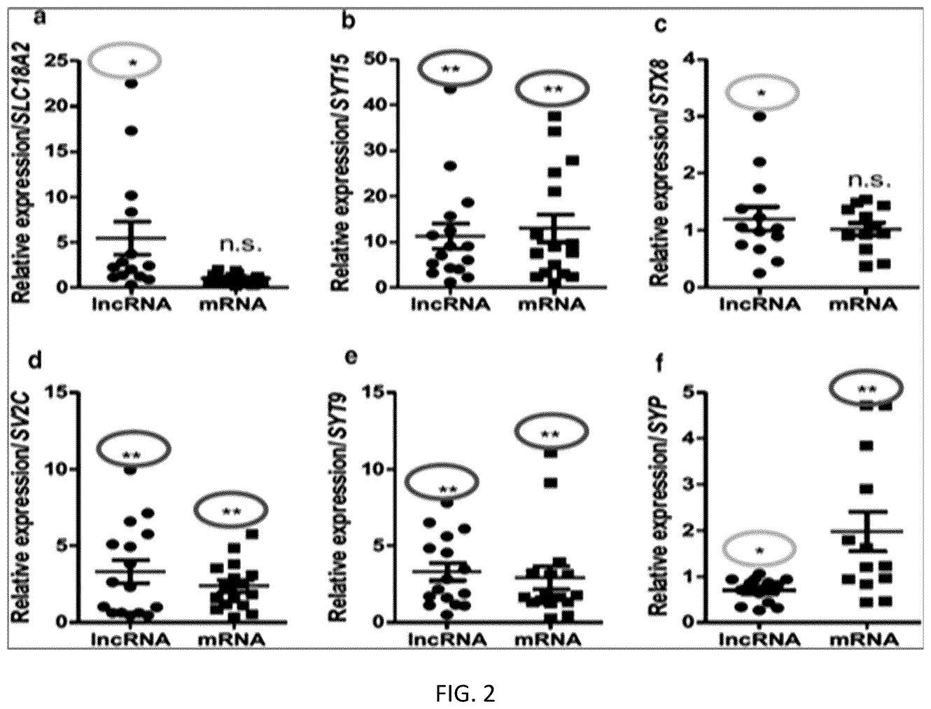

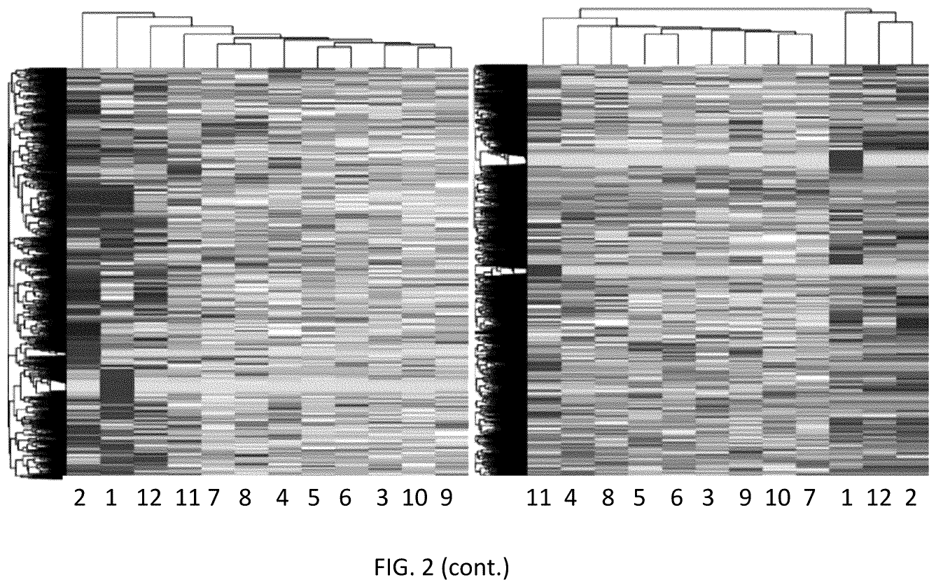

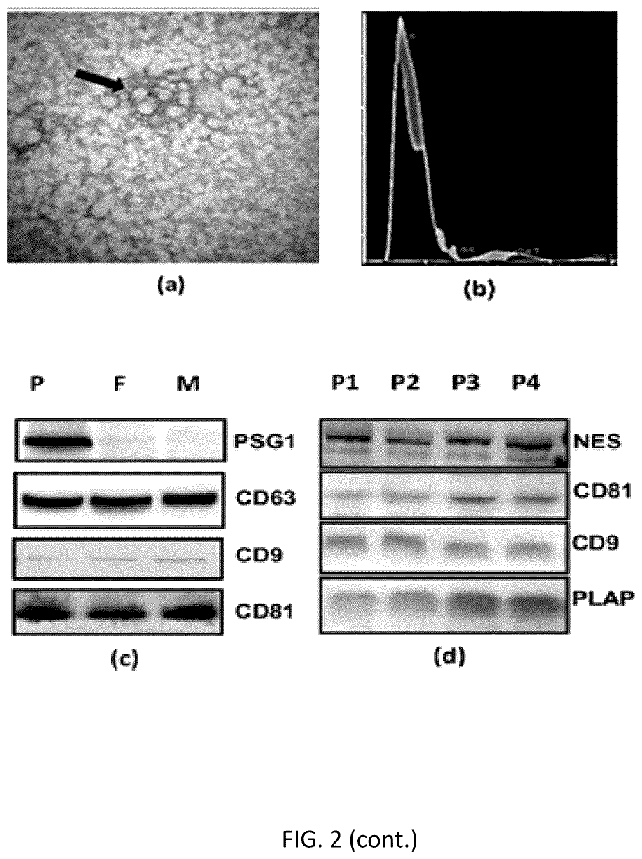

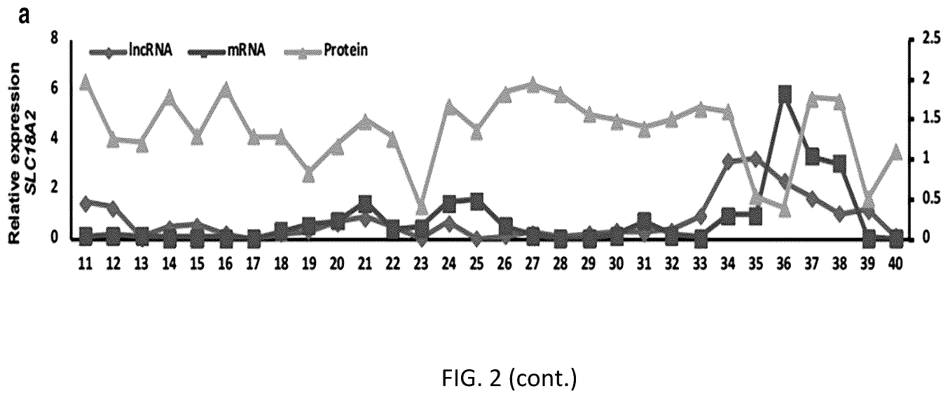

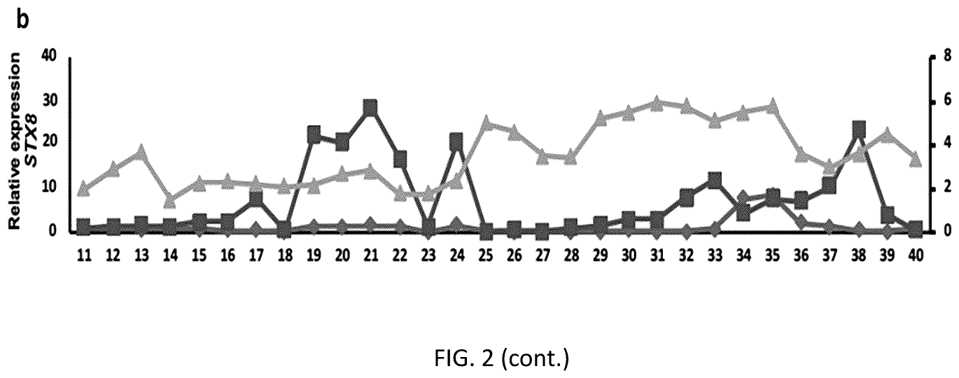

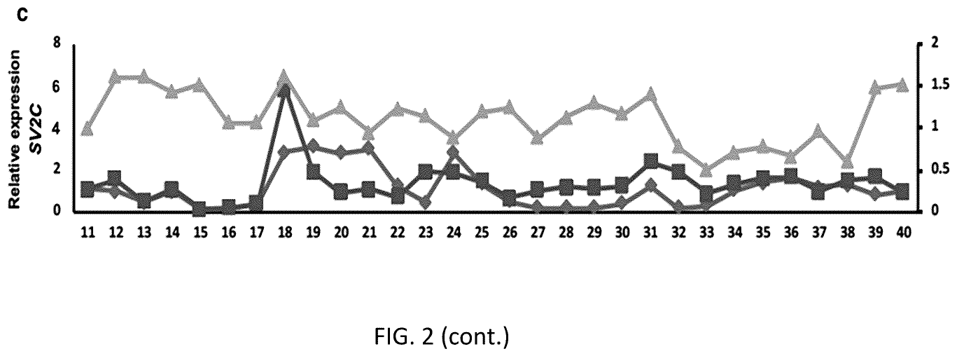

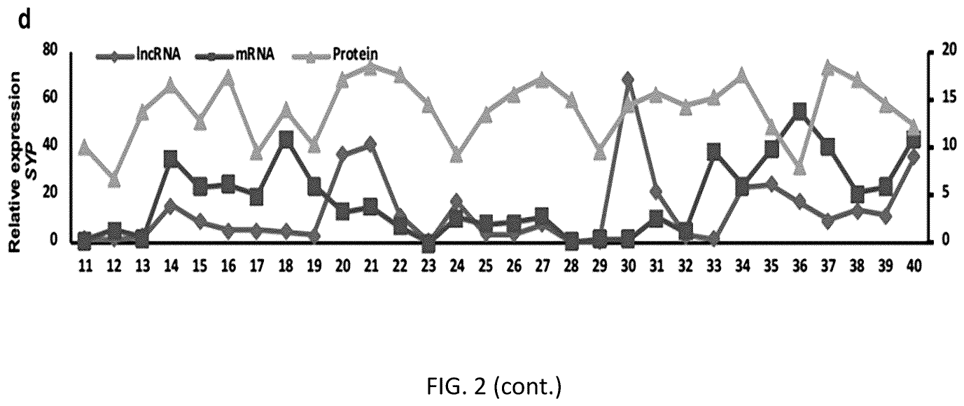

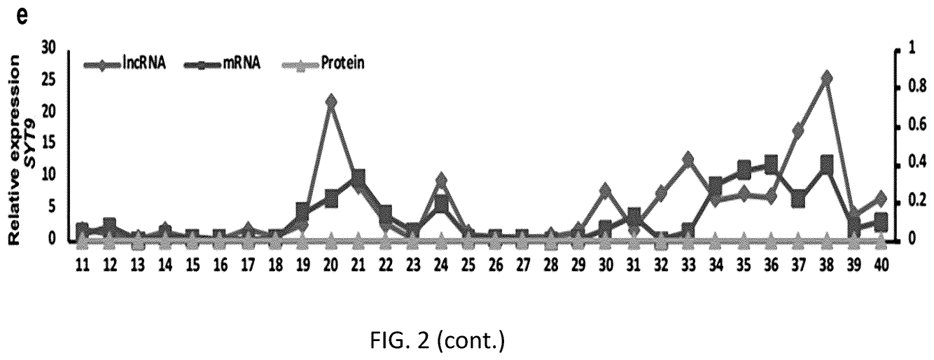

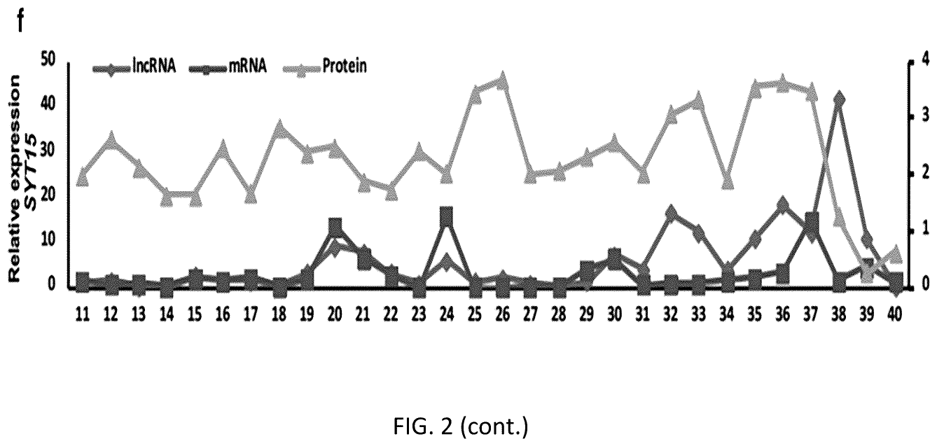

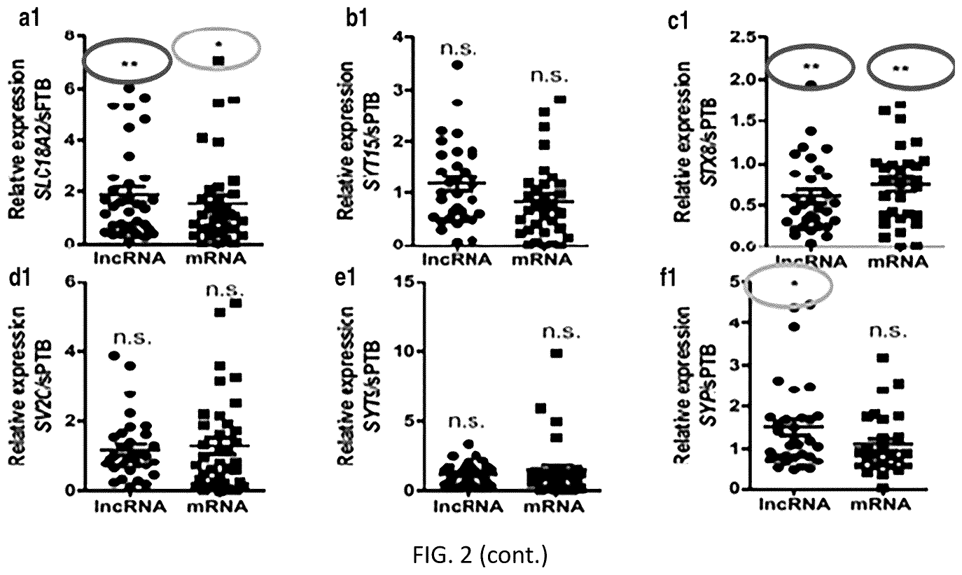

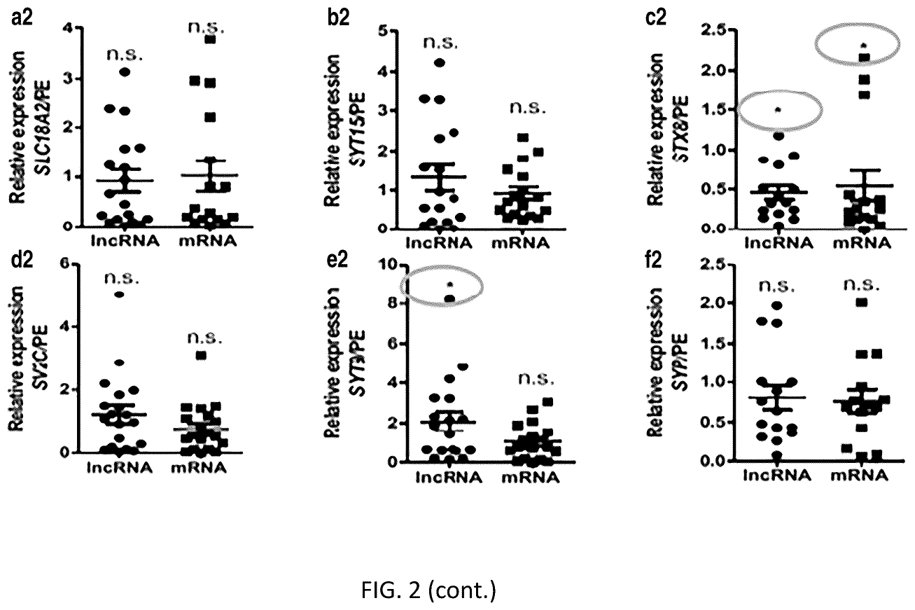

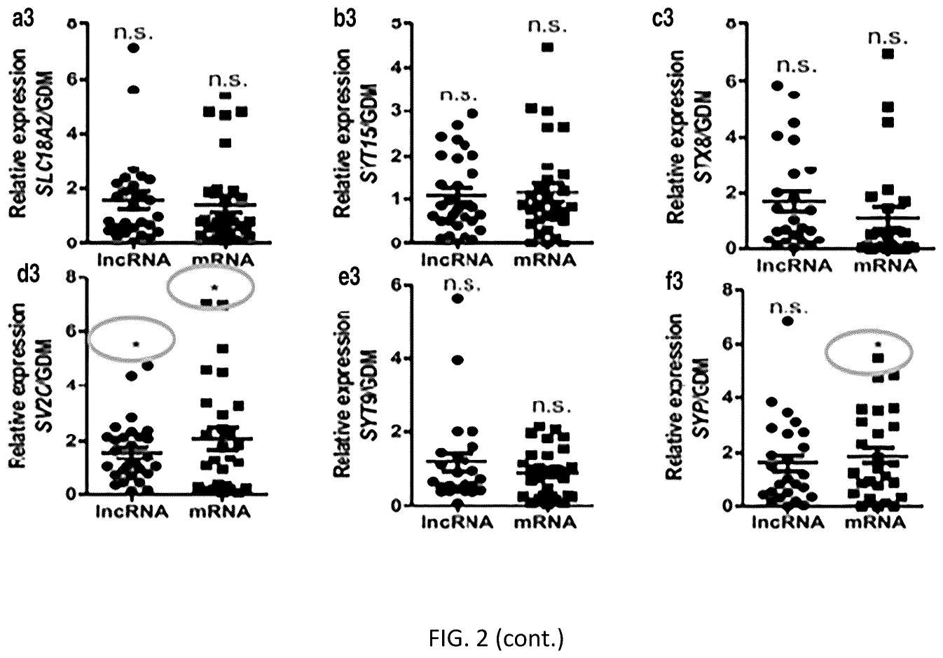

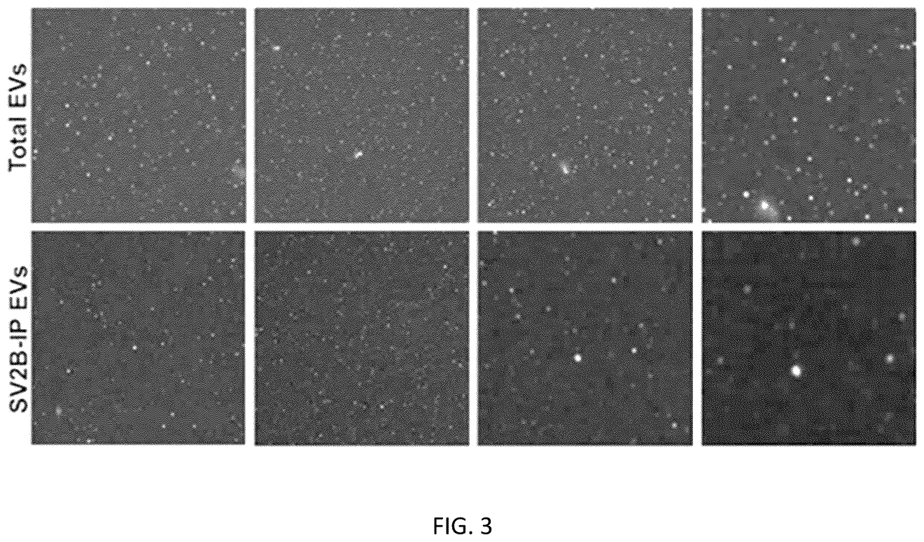

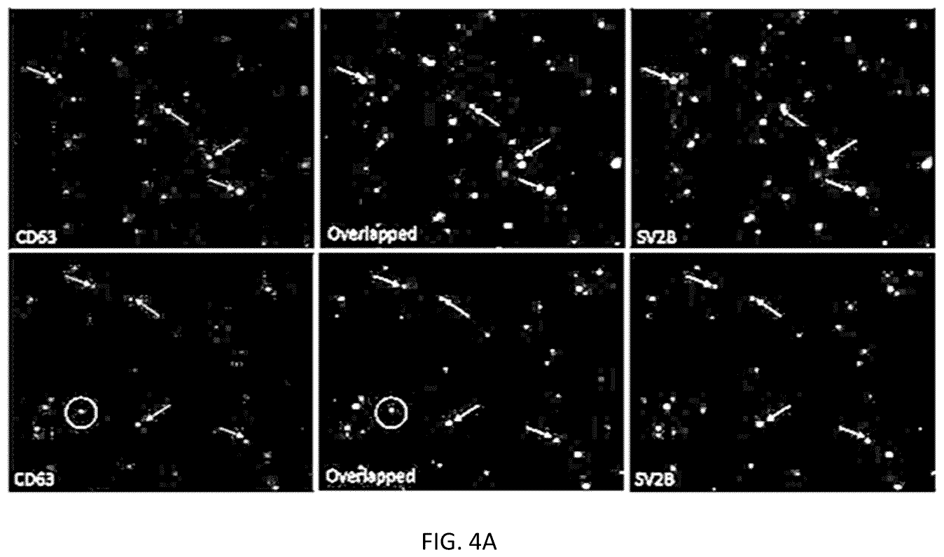

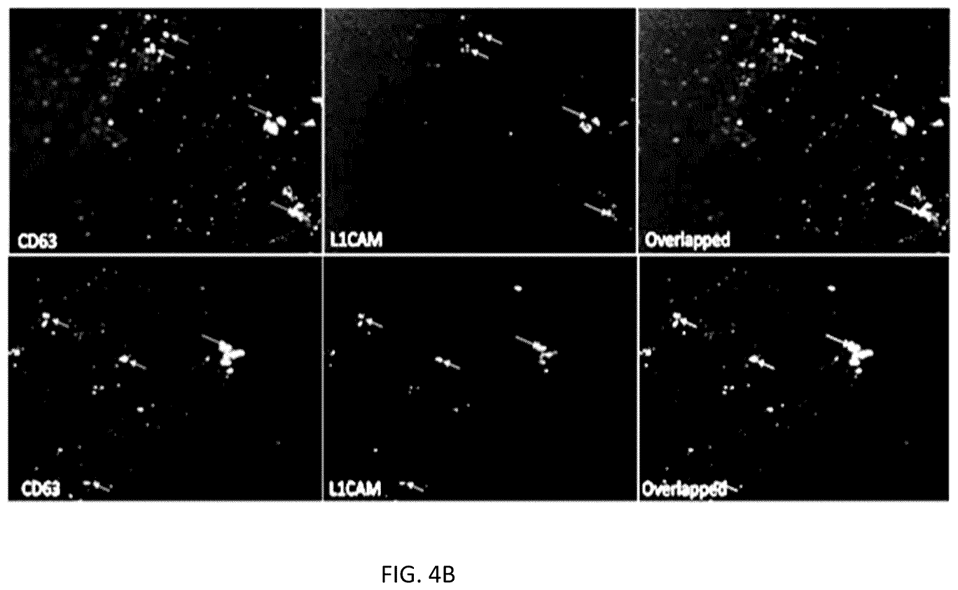

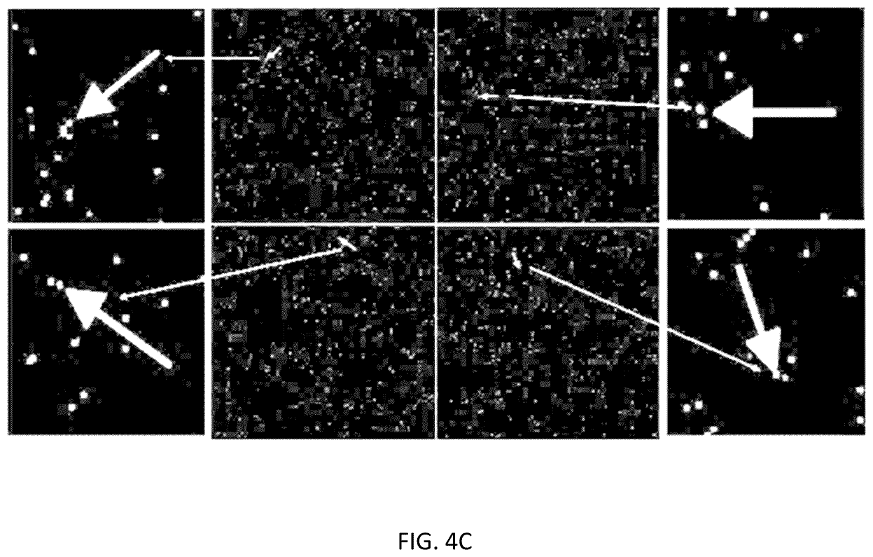

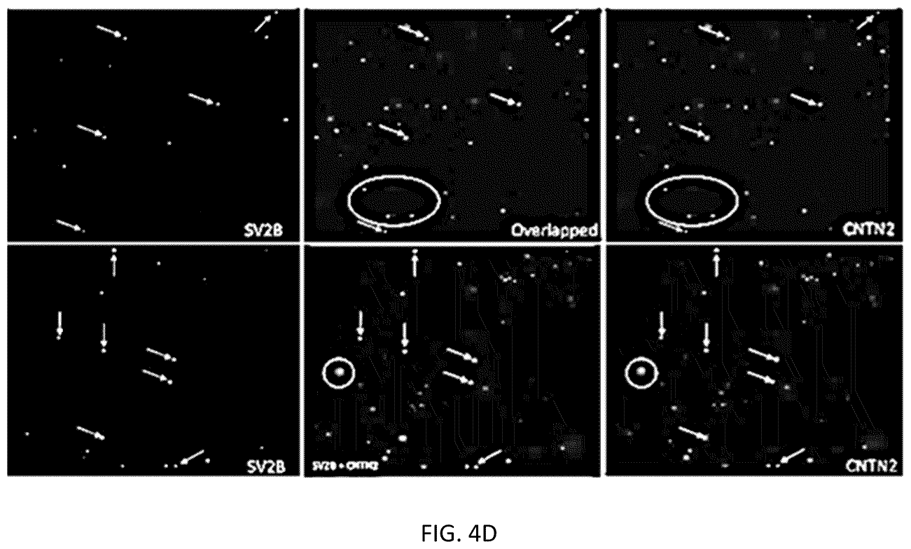

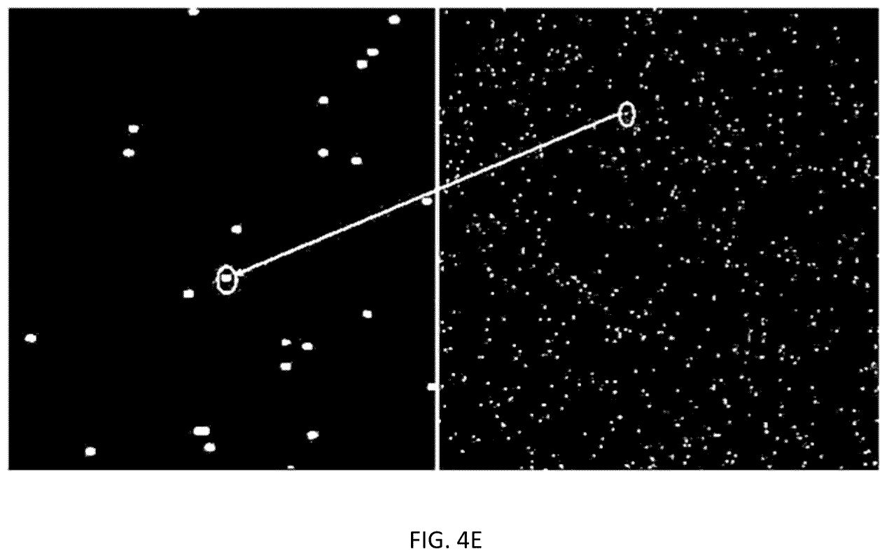

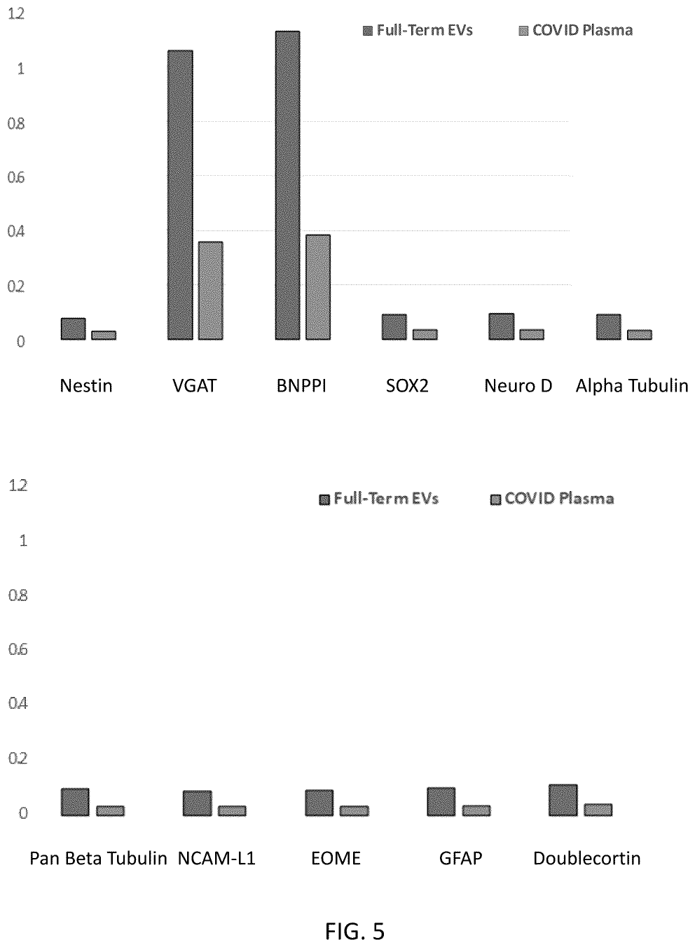

Referring to the figures, wherein like numerals refer to like parts throughout, there is seen in FIG. 1, a process 10 for immunoprecipitation of isolating fetal neuronal EVs-EXs that enables a quantification of the molecules encapsulated within the vesicles.

More specifically, process 10 starts with a sample of material blood 12 and includes a two-step procedure that involves a first step 14 where an antibody against pregnancy-specific beta-1-glycoprotein 1 (PSG1) is used to isolate fetal-originated EVs-EXs from placental syncytiotrophoblasts to obtain placenta-specific EVs-EXs that are of fetal origin circulating in maternal blood.

For example, a polymer-based approach for isolating extracellular vesicles may be used, such as the ExoQuick ULTRA EV Isolation Kit available from System Biosciences, Inc. of Mountainview, CA (Cat #EQULTRA-20A-1), along with commercially available antibodies against pregnancy-specific beta-1-glycoprotein 1 (PSG1), to isolate fetal-originated EVs-EXs from placental syncytiotrophoblasts to obtain fetal origin EVs-EXs from maternal blood. The fetal-originated EVs-EXs are resuspended in a final volume of 250 μL dH₂O and stored at −80° C.

In a second step 16, a second antibody is used to purify neuronal EVs-EXs from the placental EVs-EXs. This antibody is specifically against SV2B, a member of the synaptic vesicle proteins 2 (SV2) family that is localized to synaptic vesicles and may function in the regulation of vesicle trafficking and exocytosis in neuron cells. SV2B is more reproducible and specific to synapse development and function. SV2B was demonstrated as a fetal neuronal marker by double-staining with confocal fluorescent staining.

As an example, to isolate fetal neural EVs-EXs, 10 μg of total placental EVs-EXs in 400 μL dH₂O was incubated with 50 μL of 3% Bovine Serum Albumin (BSA) (Thermo Scientific, Inc., Waltham, MA), that contains 2 μg of rabbit polyclonal IgG anti-human/mouse SV2B antibody conjugated with biotin, such as bs-11365R-Biotin available from Bioss Inc. of Woburn, MA, for 90 min at 20°° C. Then, 10 μL of a beaded resin of immobilized recombinant streptavidin protein that binds biotinylated proteins, such as Streptavidin-Plus UltraLink resin available from Pierce-Thermo Scientific, Inc., and 40 μL of 3% BSA are added to the total placental EVs-EXs and incubated continually for 60 min at 20° C. After centrifugation at 300 g for 10 min at 4° C. and removal of the supernate, the pellet is resuspended in 75 μL of 0.05 mol/L glycine-HCl (pH 3.0), incubated at 4° C. for 10 min, and recentrifuged at 4,000 g for 10 min at 4° C. The supernate is placed in a new 1.7 mL Eppendorf tube and mixed with 5 μL of 1 mol/L Tris-HCl (pH 8.0) and 20 μL of 3% BSA followed by addition of 250 μL of a complete cell lysis reagent, such as M-PER Mammalian protein extraction reagent available from Thermo Scientific, Inc., that contains protease and phosphatase inhibitors to purify the fetal neural EVs-EXs. The purified fetal neural EVs-EXs are aliquoted in 50 μL prior to storage at −80° C. An aliquot of 5 μg of fetal neural EVs-EXs may be subjected to functional characterization with confocal microscopy or with ELISA. EVs-EXs isolated and purified by the two-step procedure of process 10 were clearly fetal and not maternal, and were neuronal, i.e., not of other cell types.

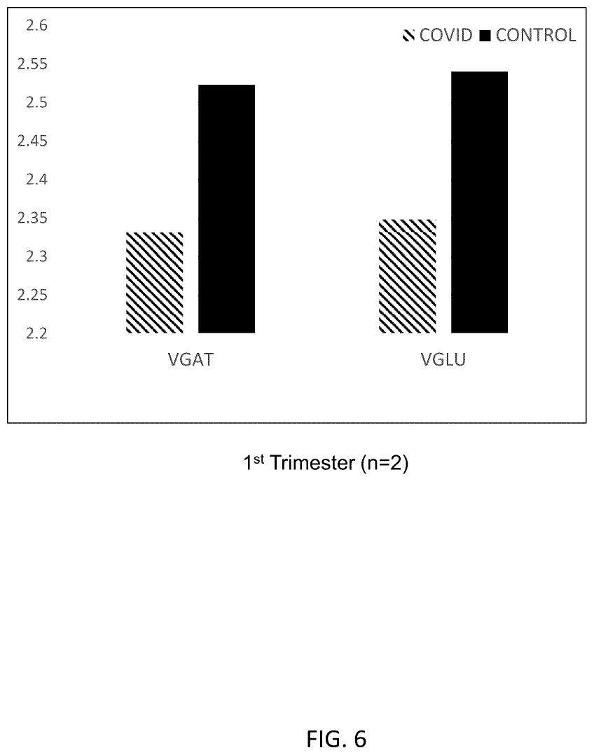

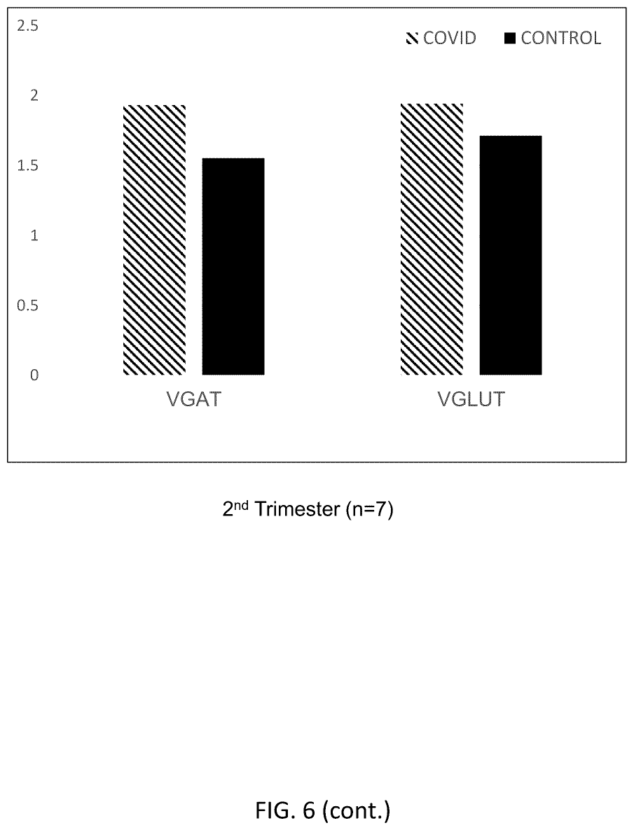

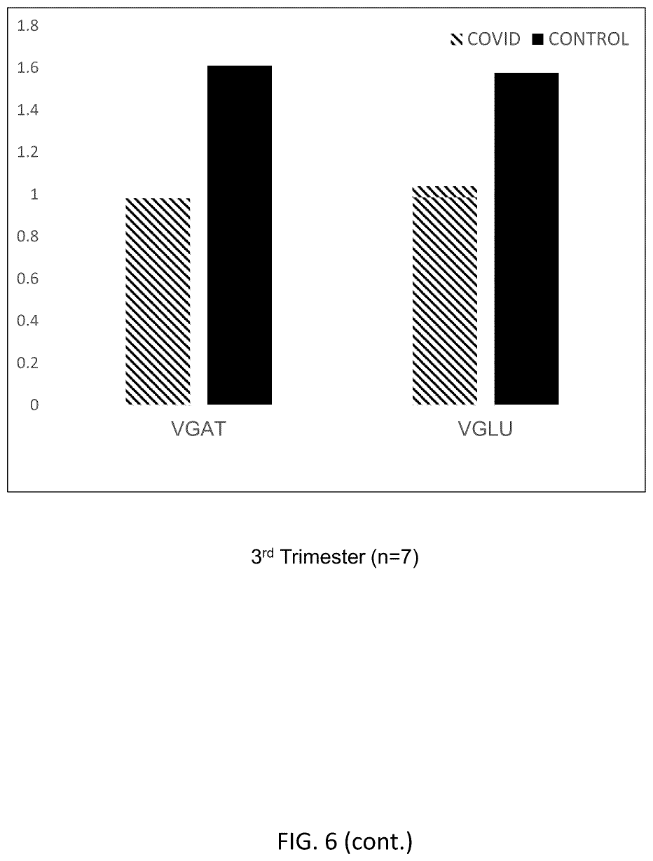

Testing showed that SV2B-immunoprecipitated fetal neural EVs-EXs according to the present invention may be used for quantitative measurement to determine synaptic function and synapse development, which are related to developmental disabilities, and thus opens up new possibilities for evaluating neuron function related to neurotransmitter release in various pregnancy scenarios. For example, obtaining fetal neuronal EVs-EXs allows for the assessment of crucial fetal neuronal synapse development by quantitating presynaptic vesicle molecules. In addition, measuring the fetal neuronal EVs-EXs at various timepoints of pregnancy (as demonstrated in FIG. 6) with a non-invasive approach, allows for in vivo monitoring of fetal brain development during the entire pregnancy period dynamically-from the first trimester to the time of birth-in a real-time fashion.

Process 10 also demonstrated that the pathological impact of viral infection in COVID-infected pregnancies reduced gene expression of vesicular inhibitory amino acid transporter (VGAT), which is a protein involved in gamma-aminobutyric acid (GABA) and glycine uptake into synaptic vesicles, and of vesicular glutamate transporter 2 (VGLUT2), which is involved in neurotransmitter loading into synaptic vesicles. This dysregulation may disrupt the delicate balance of excitatory and inhibitory neuronal signaling, potentially contributing to autism spectrum disorders and other neurodevelopmental disabilities. Additionally, the present invention may be used to measure neuronal markers L1 cell adhesion molecule (L1CAM), synaptotagmin 1 (SYT1), synaptic vesicle glyocprotein 2 (SV2B), and contactin 2 (CNTN2) in pregnancies with a history of preterm deliveries. This pioneering assessment of fetal neuronal EVs-EXs has the potential to seamlessly integrate into clinical practice, aiding in the identification of fetuses at risk of developmental disabilities. The present invention thus opens up new possibilities for evaluating neuron function related to neurotransmitter release in various pregnancy scenarios.

What is claimed is:

1. A method of obtaining fetal neuronal extracellular vesicles from a maternal blood sample, comprising the steps of:
isolating any fetal-originated extracellular vesicles in the material blood sample originating from placental syncytiotrophoblasts with an amount of antibodies specific to pregnancy-specific beta-1-glycoprotein 1 (PSG1); and
separating any neuronal extracellular vesicles from any placental extracellular vesicles in the isolated fetal-originated extracellular vesicles by treating the isolated fetal-originated extracellular vesicles with an amount of antibodies specific to synaptic vesicle glycoprotein 2B (SV2B).

2. The method of claim 1, further comprising resuspending the fetal-originated extracellular vesicles in water prior to separating the neuronal extracellular vesicles from the placental extracellular vesicles.

3. The method of claim 1, wherein the amount of antibodies specific to synaptic vesicle glycoprotein 2B (SV2B) comprise rabbit polyclonal IgG anti-human/mouse SV2B antibodies conjugated with biotin.

4. The method of claim 3, wherein separating the neuronal extracellular vesicles from the placental extracellular vesicles includes adding a beaded resin that binds any biotinylated proteins.

5. The method of claim 4, wherein separating the neuronal extracellular vesicles from the placental extracellular vesicles includes centrifuging the beaded resin and removing a first supernate to provide a first pellet.

6. The method of claim 5, wherein separating the neuronal extracellular vesicles from the placental extracellular vesicles includes resuspending the first pellet in glycine-HCl and then centrifuging to provide a second supernate.

7. The method of claim 6, wherein isolating any fetal-originated extracellular vesicles in the maternal blood sample includes mixing the second supernate with a cell lysis reagent containing protease and phosphatase inhibitors to provide an amount of purified fetal neural extracellular vesicles.

8. A method of monitoring fetal brain development, comprising the steps of:
isolating fetal neural extracellular vesicles from a sample of maternal blood by treating the sample of material blood with an amount of antibodies specific to pregnancy-specific beta-1-glycoprotein 1 (PSG1) to obtain isolated fetal-originated extracellular vesicles and then separating any neuronal extracellular vesicles from any placental extracellular vesicles in the isolated fetal-originated extracellular vesicles by treating the isolated fetal-originated extracellular vesicles with an amount of antibodies specific to synaptic vesicle glycoprotein 2B (SV2B);
measuring a marker of the fetal neural extracellular vesicles; and
repeating the steps of isolating the fetal neural extracellular vesicles and measuring the marker of the fetal neural extracellular vesicles repeatedly over a predetermined period of time.

9. The method of claim 8, wherein the predetermined period of time commences during the first trimester and ends at birth.

10. The method of claim 8, wherein the marker is an amount of expression of a vesicular inhibitory amino acid transporter (VGAT) gene.

11. The method of claim 8, wherein the marker is an amount of expression of a vesicular glutamate transporter 2 (VGLUT2) gene.

12. The method of claim 8, wherein the marker is an amount of expression of a gene selected from the group consisting of a L1 cell adhesion molecule (L1CAM) gene, a synaptotagmin 1 (SYT1) gene, a synaptic vesicle glyocprotein 2 (SV2B) gene, and a contactin 2(CNTN2) gene.

13. The method of claim 8, wherein the amount of antibodies specific to synaptic vesicle glycoprotein 2B (SV2B) comprise rabbit polyclonal IgG anti-human/mouse SV2B antibodies conjugated with biotin.

14. The method of claim 13, wherein separating the neuronal extracellular vesicles from the placental extracellular vesicles includes adding a beaded resin that binds any biotinylated proteins.

15. The method of claim 14, wherein separating the neuronal extracellular vesicles from the placental extracellular vesicles includes centrifuging the beaded resin and removing a first supernate to provide a first pellet.

16. The method of claim 15, wherein separating the neuronal extracellular vesicles from the placental extracellular vesicles includes resuspending the pellet in glycine-HCl and then centrifuging to provide a second supernate.

17. The method of claim 16, wherein isolating any fetal-originated extracellular vesicles in the blood sample includes mixing the second supernate with a cell lysis reagent containing protease and phosphatase inhibitors to provide an amount of purified fetal neural extracellular vesicles.
